(19) United States
(12) Patent Application Publication
(10) Pub. No.: US 2007/0260190 A1
(43) Pub. Date: Nov. 8, 2007

(54) Safety device for intravenous injection unit

(76) Inventor: Hui-Chiang Lin, Hemei Township (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/444,325

(22) Filed: Jun. 1, 2006

(30) Foreign Application Priority Data
May 2, 2006 (TW) 095207488

(51) Int. Cl. A61M 5/32

(52) U.S. Cl. 604/192

(57) ABSTRACT

An intravenous injection unit includes a needle set which has a first end connected with a hose and a needle is connected to a second end of the needle set. A protrusion and a wing extend from an outer periphery of the needle set, and a sleeve is movably mounted to the needle set and the wing and the protrusion are movably engaged with the slot. A plurality of stop plates extend from an inner periphery of the first end of the sleeve inclinedly and inward so that the sleeve is moved toward the needle set after use till the protrusion moves over and is engaged with the stop plates, such that the needle is received in the sleeve and the needle set cannot be moved again.

### FIELD OF THE INVENTION

The present invention relates to a safety device for intravenous injection unit and the sleeve of the safety device can be slidably along the hose and receives the needle therein after use.

### BACKGROUND OF THE INVENTION

A conventional intravenous injection device 80 is shown in FIGS. 8 and 9, and generally includes a sleeve 81 with two open ends and a slot 85 is defined through a wall of the sleeve 80. Two teeth 851 extend from the two separated sides defining the slot 85 and are located close to a first end of the sleeve 81. A recess is defined in the second end of the sleeve 81 and has serrated periphery 86. A needle set 82 has a hose 83 connected to a first end thereof and a needle is connected to a second end of the set 82. The needle set 82 extends through the sleeve 81 from the first end of the sleeve 81 so that the needle extends out from the second end of the sleeve 81. A wing 84 is connected to the set 82 and extends out from the slot 85 so that the wing 84 can be taped on the patient's arm to position the needle set 82. After injection, the nurse simply pushes the sleeve 81 toward the needle so that the needle is received in the sleeve 81. The hose 83 is then engaged with the recess and positioned by the serrated periphery 86, and the teeth 851 prevent the needle set 82 from being disengaged from the sleeve 81. However, the hose 83 has a smooth outer periphery which might slip away from the serrated periphery 86 and once the hose 83 is not well positioned, the sleeve 81 can be slid and the needle of the needle set 82 may extend out from the second end of the sleeve 81 again.

The present invention intends to provide a safety device of intravenous injection unit wherein the needle set can be secured after use so that the needle is received in the sleeve and cannot extend out from the sleeve again.

### SUMMARY OF THE INVENTION

The present invention relates to an intravenous injection unit which comprises a needle set including a connection end on a first end thereof and a needle is connected to a second end of the needle set. A hose has one end connected with the connection end of the needle set. A protrusion and a wing extending from an outer periphery of the needle set. A sleeve has a slot longitudinally defined through a wall thereof and a plurality of stop plates extend from an inner periphery of the first end of the sleeve inclinedly and inward. The wing and the protrusion are movably engaged with the slot and the protrusion is moved over and engaged with the stop plates when the sleeve is moved toward the needle set after use.

The primary object of the present invention is to provide a safety device for intravenous injection unit, wherein the needle can be well positioned in the sleeve after use.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show the safety device for intravenous injection unit of the present invention;

FIG. 2 is an exploded view to show the safety device for intravenous injection unit of the present invention;

FIG. 3 is a cross sectional view of the safety device for intravenous injection unit of the present invention, wherein the intravenous injection unit has not yet been used;

FIG. 4 is a cross sectional view of the safety device for intravenous injection unit of the present invention, wherein the sleeve is moved to receive the needle therein;

FIG. 5 shows that the number of the wings can be two;

FIG. 6 is a cross sectional view of the safety device for intravenous injection unit in FIG. 5, wherein the sleeve is moved to receive the needle therein;

FIG. 7 shows yet another embodiment wherein a side input tube is connected to the needle set of the present invention;

FIG. 8 is an exploded view to show a conventional intravenous injection unit, and

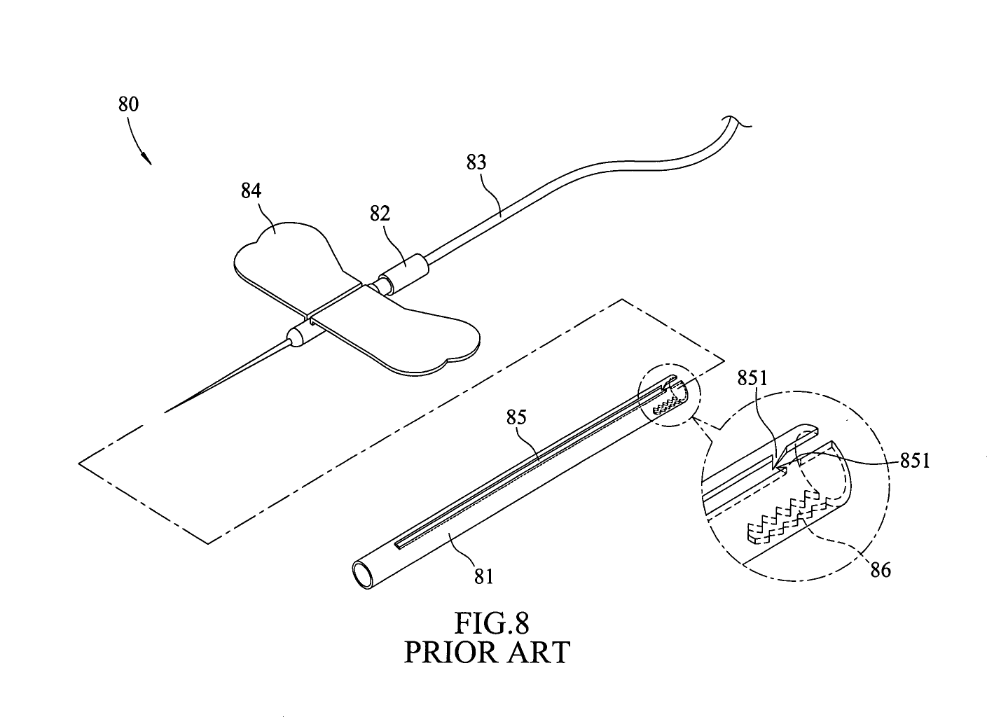

FIG. 9 is a perspective view of the conventional intravenous injection unit.

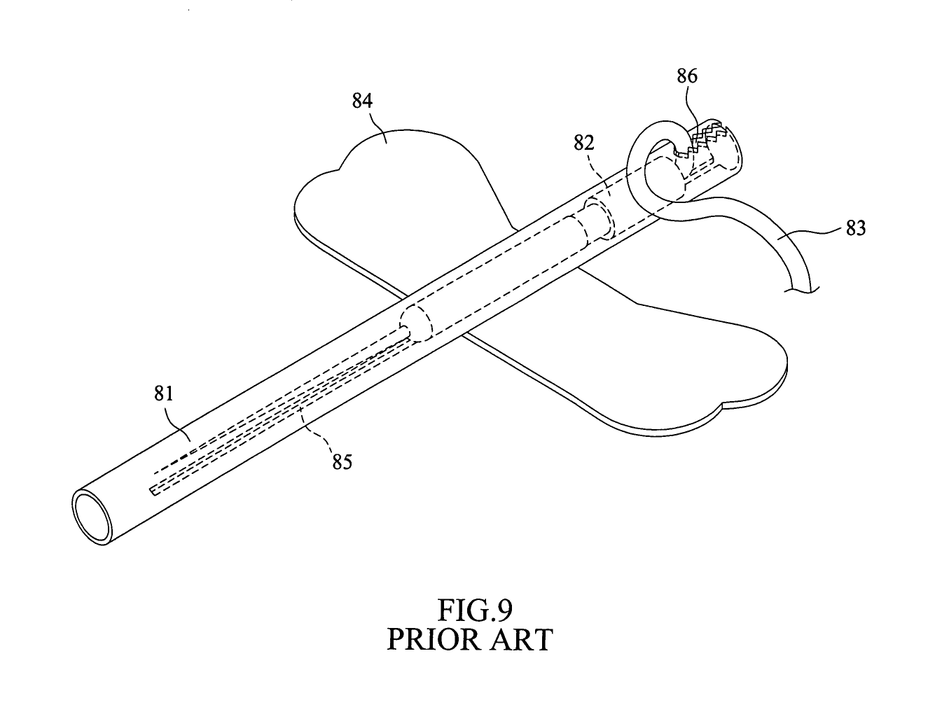

### DETAILED DESCRIPTION OF THE PREFERRED EMBODIMENT

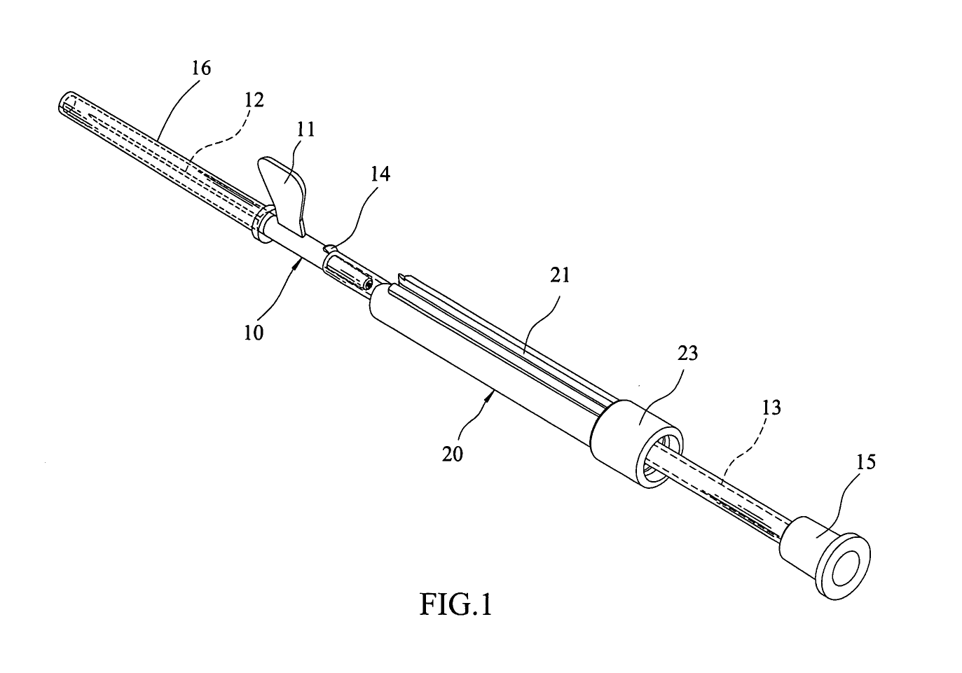

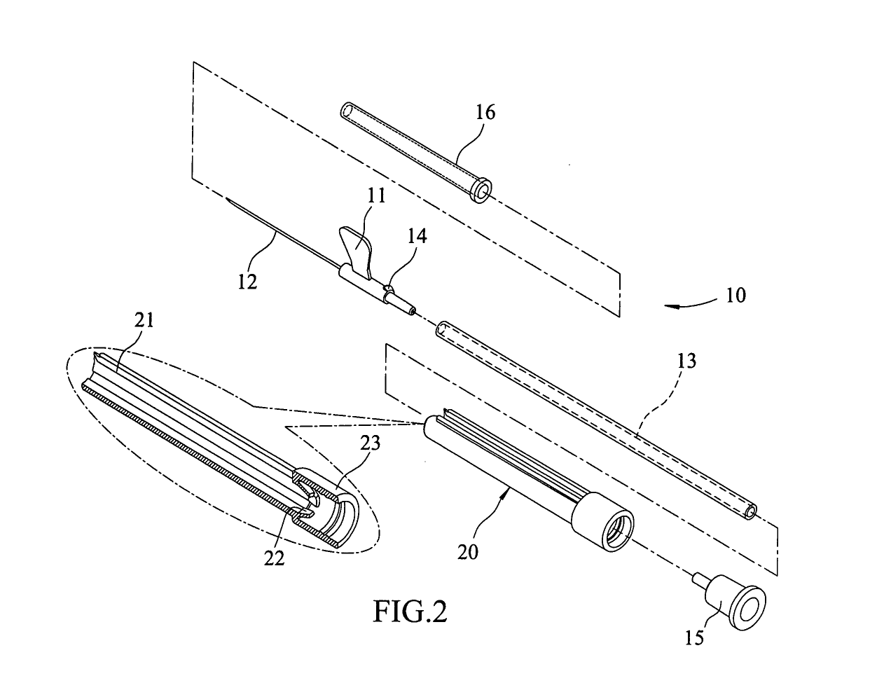

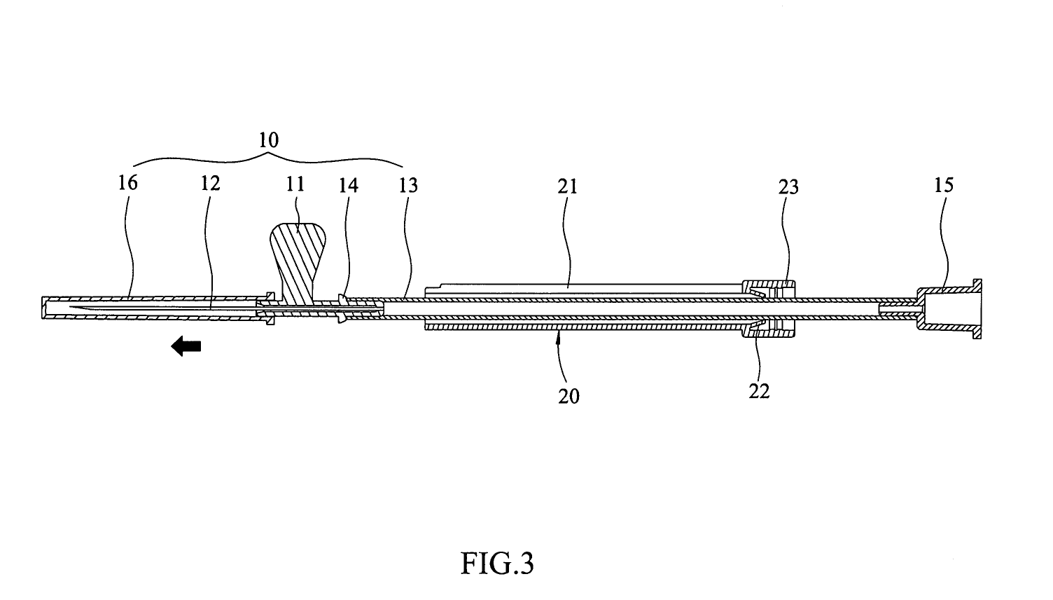

Referring to FIGS. 1 to 3, the intravenous injection unit of the present invention comprises a needle set 10 including a connection end on a first end thereof so as to be connected with an end of a hose 13, and a needle 12 is connected to a second end of the needle set 10. A protrusion 14 and a wing 11 extend from an outer periphery of the needle set 10 at a distance. A connector 15 which is connected to the other end of the hose 13 so that medicine can be supplied to the hose 13 via the connector 15.

A sleeve 20 has a slot 21 longitudinally defined through a wall thereof and a head 23 is connected to a first end of the sleeve 20. The slot 21 is terminated before the head 23 and communicates with a second end of the sleeve 20. A plurality of stop plates 22 extending from an inner periphery of the first end of the sleeve 20 inclinedly and inward. Each of the stop plates 22 is a thin and flexible plate and can be pushed outward slightly.

The wing 11 and the protrusion 14 are movably engaged with the slot 21 and the hose 13 extends out from the first end of the sleeve 20. A cover 16 is removably connected to the needle set 10 and the needle 12 is received in the cover 16. When in use, the cover 16 is first removed and the wing 11 is taped to the patient's limb. Medicine is supplied via the hose 13 and enters the patient's vein via the needle 12.

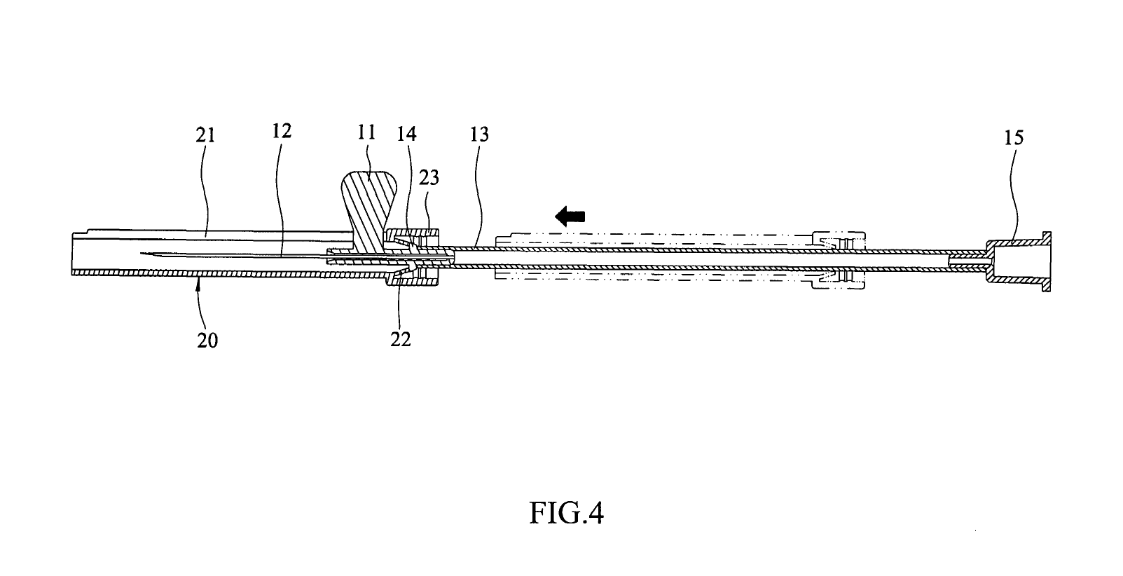

After use, as shown in FIG. 4, the sleeve 20 is moved toward the needle set 10 until the protrusion 14 moves over and is engaged with the stop plates 22. By the engagement of the stop plates 22 and the protrusion 14, the sleeve 20 cannot be pulled backward so as to ensure that the needle 12 is received in the sleeve 20.

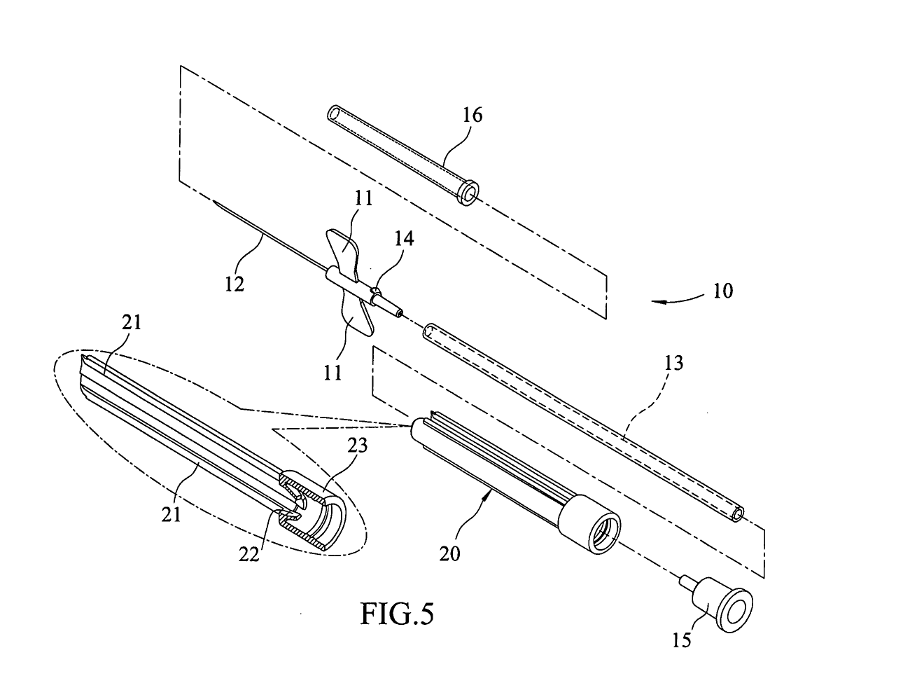

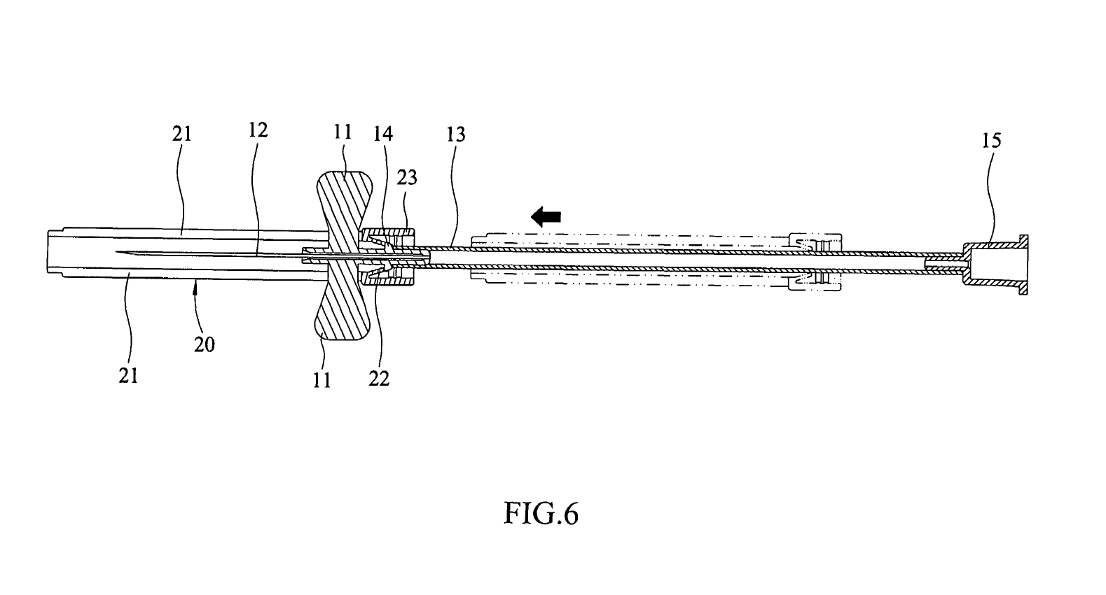

FIGS. 5 and 6 show that the number of the wings 11 can be two so that there are two slots 21 defined in the sleeve 20 so that the two wings 11 can movably extend through the two slots 21.

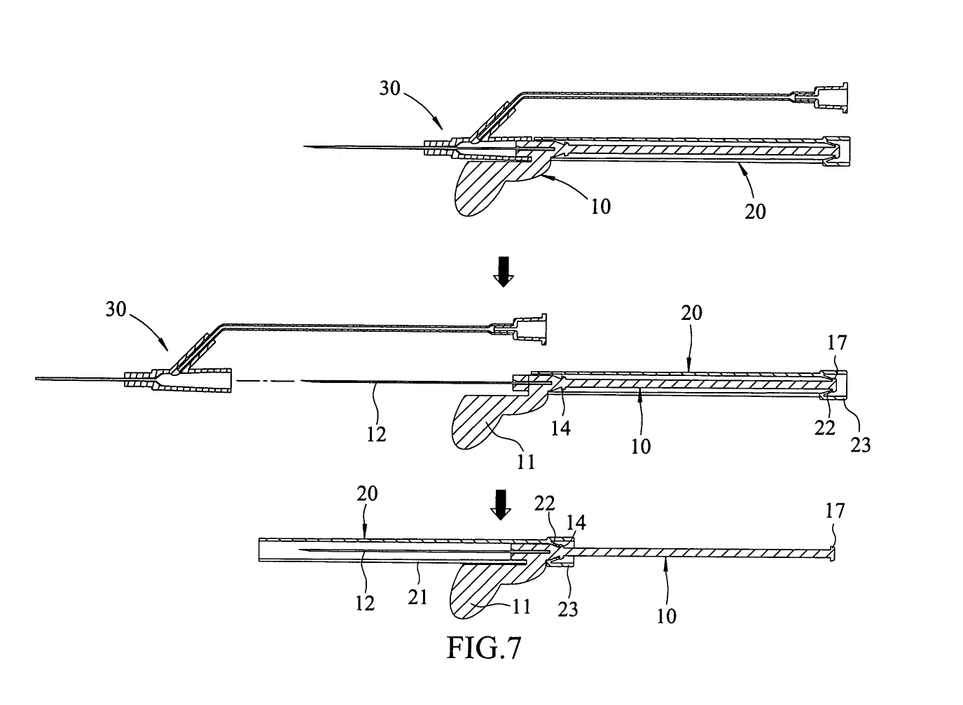

FIG. 7 shows yet another embodiment wherein a side input tube 30 is connected to the needle set 10 and communicates with the needle 12. Therefore, medicine can be sent to the needle 12 via the side input tube 30. An enlarged head 17 is connected to the first end of the needle set 10 and engaged with the stop plates 22 so as to ensure the sleeve 20 cannot be pulled away from the needle set 10 to disengage the needle set 10 from the sleeve 20.

While we have shown and described the embodiment in accordance with the present invention, it should be clear to those skilled in the art that further embodiments may be made without departing from the scope of the present invention.

What is claimed is:

1. An intravenous injection unit, comprising:
a needle set including a connection end on a first end thereof and a needle connected to a second end of the needle set, a hose having one end connected with the connection end of the needle set, a protrusion and a wing extending from an outer periphery of the needle set, and
a sleeve having a slot longitudinally defined through a wall thereof and a plurality of stop plates extending from an inner periphery of the first end of the sleeve inclinedly and inward, the wing and the protrusion movably engaged with the slot and the protrusion being moved over and being engaged with the stop plates when the sleeve is moved toward the needle set.

2. The device as claimed in claim 1, wherein a head is connected to the first end of the sleeve and the slot is terminated before the head.

3. The device as claimed in claim 1, wherein each of the stop plates extends inward from the inner periphery of the first end of the sleeve and is pushed outward slightly.

4. The device as claimed in claim 1 further comprising a connector which is connected to the other end of the hose.

5. The device as claimed in claim 1 further comprising a cover which is removably connected to the needle set and the needle is received in the cover.

6. The device as claimed in claim 1, wherein a side input tube is connected to the needle set and communicates with the needle, an enlarged head is connected to the first end of the needle set and engaged with the stop plates.
